SEQUENCE LISTING (4 sequences)

SEQ ID NO: 1 - protein, 518 aa, Homo sapiens
ARQAAWREGAGLRGREGARAGGNRTPPASMAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKDYKCKVSNKALPAPMQKTISKAKGQPREPQVYTLPPSRDELTKNQVSLTCLVKGFYPRHIAVEWESNGQPENNYKTTPPVLDSDGSFFLYSKLTVDKSRWQQGNVFSCSVMHEALHNHYTQKSLSLSPGK

SEQ ID NO: 2 - DNA, 1557 bp, Homo sapien
gcgaggcaggcagcctggagagaaggcgctgggctgcgagggcgcgagggcgcgagggcagggggcaaccggaccccgcccgcatccatggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggactacaagtgcaaggtctccaacaaagccctcccagcccccatgcagaaaaccatctccaaagccaaagggcagccccgagaaccacaggtgtacaccctgcccccatcccgggatgagctgaccaagaaccaggtcagcctgacctgcctggtcaaaggcttctatcccaggcacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 3 - protein, 258 aa, Rattus Rattus
MAPAALWVALVVELQLWATGHTVPAKVVLTPYKPEPGNQCQISQEYYDKKAQMCCAKCPPGQYAKHFCNKTSDTVCADCAAGMFTQVWNHLHTCLSCSSSCSDDQVETHNCTKKQNRVCACNADSYCALKLHSGNCRQCMKLSKCGPGFGVARSRTSNGNVICSACAPGTFSDTTSSTDVCRPHGICSILAIPGNASTDAVCASESPTPSAVPRTIYVSQPEPTRSQPMDQEPGPSQTPHIPVSLGSTPIIEPSITGG

SEQ ID NO: 4 - DNA, 774 bp, Rattus rattus
atggcgcccgccgccctctgggtcgcgctggtcgtcgaactgcagctgtgggccaccgggcacacagtgcccgccaaggttgtcttgacaccctacaagccagaacctgggaaccagtgccagatctcacaggagtactatgacaagaaggctcagatgtgctgtgctaagtgtccccctggccagtatgcaaaacacttctgcaacaagacttcagacaccgtgtgtgcggactgtgcggcaggcatgtttacccaggtctggaaccatctgcatacatgcctgagctgcagttcttcctgtagtgatgaccaggtggagacccacaactgcactaaaaaacagaaccgagtgtgtgcttgcaacgctgacagttactgtgccttgaaattgcattctgggaactgtcgacagtgcatgaagctgagcaagtgtggccctggcttcggagtggcccgttcaagaacctcaaatggaaacgtgatatgcagtgcctgtgccccagggacgttctctgacaccacatcatccacagatgtgtgcaggccccacggcatttgtagcatcctggctattcctggaaatgcaagcacggatgcagtctgtgcatccgagtccccaactccaagcgctgttccaaggacaatctacgtatctcagccagagcccacaagatcccagcccatggatcaagagccagggcctagccaaactccacacatccctgtgtccttgggttcaacccccatcattgaaccaagcatcacgggtggg

(19) United States
(12) Patent Application Publication
(10) Pub. No.: US 2008/0175845 A1
(43) Pub. Date: Jul. 24, 2008

(54) Methods for treating target joints in inflammatory arthritis using AAV vectors encoding a TNF antagonist

(75) Inventor: Pervin Anklesaria, Edmonds, WA (US)

Correspondence Address: MORRISON & FOERSTER LLP, 755 PAGE MILL RD, PALO ALTO, CA, 94304-1018, US

(73) Assignee: Targeted Genetics Corporation, Seattle, WA (US)

(21) Appl. No.: 11/818,790

(22) Filed: Jun. 14, 2007

Related U.S. Application Data
(60) Provisional application No. 60/813,916, filed on Jun. 15, 2006.

(51) Int. Cl. A61K 39/395; A61K 31/70; A61K 38/00

(52) U.S. Cl. 424/141.1; 514/44; 514/12

(57) ABSTRACT

The present invention provides methods for treating inflammatory arthritis in an individual, comprising administering to the individual an effective amount of AAV (rAAV) vector comprising a polynucleotide encoding a pro-inflammatory cytokine antagonist, wherein the individual is being treated systemically with a polypeptide pro-inflammatory antagonist but still has one or more persistently symptomatic joints.

### CROSS-REFERENCE TO RELATED APPLICATIONS

This application claims the priority benefit of provisional patent application U.S. Ser. Nos. 60/813,916, filed Jun. 15, 2006, which is incorporated herein in its entirety by reference.

### FIELD OF INVENTION

This invention relates to methods for the treatment of arthritis or arthritic syndromes. More specifically, the invention relates to a method of treating an individual with persistently symptomatic arthritic joints, wherein the individual is being treated systemically with polypeptide pro-inflammatory antagonists, by administering to the persistently symptomatic joint an adeno-associated (AAV) virus vector containing a polynucleotide encoding a pro-inflammatory cytokine antagonist.

### STATEMENT OF RIGHTS TO INVENTIONS MADE UNDER FEDERALLY SPONSORED RESEARCH

Not Applicable.

### BACKGROUND

Tumor necrosis factor-.alpha. (TNF.alpha.) and tumor necrosis factor-.beta. (TNF.beta.) are homologous multifunctional cytokines; the great similarities in structural and functional characteristics of which have resulted in their collective description as tumor necrosis factor or “TNF.” Activities generally ascribed to TNF include: release of other cytokines including IL-1, IL-6, GM-CSF, and IL-10, induction of chemokines, increase in adhesion molecules, growth of blood vessels, release of tissue destructive enzymes and activation of T cells. See, for example, Feldmann et al., 1997, Adv. Immunol., 64:283-350, Nawroth et al., 1986, J. Exp. Med., 163:1363-1375; Moser et al., 1989, J. Clin. Invest., 83:444-455; Shingu et al., 1993, Clin. Exp. Immunol. 94:145-149; MacNaul et al., 1992, Matrix Suppl., 1:198-199; and Ahmadzadeh et al., 1990, Clin. Exp. Rheumatol. 8:387-391. All of these activities can serve to enhance an inflammatory response.

TNF initiates its biological effect through its interaction with specific, cell surface receptors on TNF-responsive cells. There are two distinct forms of the cell surface tumor necrosis factor receptor (TNFR), designated p75 (or Type II) and p55 (or Type I) (Smith et al., 1990, Science 248:1019-1023; Loetscher et al., 1990, Cell 61:351-359). TNFR Type I and TNFR Type II each bind to both TNF.alpha. and TNF.beta. Soluble, truncated versions of the TNFRs with a ligand-binding domain are present in body fluids and joints (Engelmann et al., 1989, J. Biol. Chem. 264:11974-11980; Roux-Lombard et al., 1993, Arthritis Rheum. 36:485-489).

A number of disorders are associated with elevated levels of TNF, many of them of significant medical importance. Among such TNF-associated disorders are congestive heart failure, inflammatory bowel diseases (including Crohn's disease), arthritis and asthma.

Arthritis is a common crippling condition for which there are no cures and few effective therapies. Approximately one in seven people in the United States are affected by one or more forms of arthritis. Most forms of arthritis are characterized by chronic inflammation of joints resulting from infection, mechanical injury, or immunological disturbance. Rheumatoid arthritis (RA) is a chronic inflammatory disease primarily manifest in the joints by swelling, pain, stiffness, and tissue destruction (Harris, 1990, N. Engl. J. Med, 323:994-996). Systemic manifestations can include elevations in serum levels of acute phase proteins, fever, mild anemia, thrombocytosis, and granulocytosis. In affected joints, there is a synovitis characterized by hyperplasia and inflammation of the synovium with an inflammatory exudate into the joint cavity, leading to erosion of cartilage and bone.

Although rheumatoid arthritis is not directly and imminently life threatening, recent data suggest that RA results in significantly shorter lifespan, and puts an enormous toll on the both the health system, the overall economy due to lost productivity, as well as quality of life resulting from restricted mobility and activities (Schiff, 1997, Am. J. Med., 102(1A):11S-15S).

Current commonly employed therapeutics for treatment of RA fall primarily in three categories: non-steroidal anti-inflammatory drugs (NSAIDs), disease-modifying anti-rheumatic drugs (DMARDs), and immunosuppressives. NSAIDs are a large group of drugs often used as first line therapy for rheumatoid arthritis. The compounds act primarily through blockade of cyclooxygenase which catalyzes conversion of arachidonic acid to prostaglandins and thromboxanes. As a class, DMARDs, including agents such as gold, sulfasalazine, hydroxychloroquine, and D-penicillamine, are slow acting, quite toxic and there is little evidence that any of these compounds have mitigating effects on the underlying disease. NSAIDs can relieve some of the signs of inflammation and pain associated with arthritis; however, they appear to be ineffective against the immune system and in blocking progression of joint destruction and disease. Immunosuppressive agents, such as corticosteroids and methotrexate, are commonly used in the treatment of RA for suppressing the immune system and thus having an anti-inflammatory effect. However, these agents engender serious systemic toxicity which limits their use and effectiveness.

Although it is widely accepted that RA is an immune-based inflammatory disease, the antigen(s) which trigger the disease remain unknown. This has led to a large number of approaches to therapy under pre-clinical or clinical investigation which involve attempts to modulate the immune response system as a whole. Examples of several general efforts in this direction are highlighted below.

The mechanism of action of NSAIDs has been linked to blocking of cyclooxygenase, an enzyme with both an inducible and a constitutive form. As the inducible form of cyclooxygenase appears to be elevated in inflammatory disease, investigation into compounds selective for the inducible form are underway. In addition, attempts to devise vaccines to treat ongoing arthritis have been made with the use of peptide vaccines directed toward MHC class II or T cell receptor proteins. Generally, it has been proven difficult to demonstrate efficacy of vaccines administered to ongoing disease.

Much of the tissue destruction in RA appears to be due to various metalloproteinases. This group of proteases are believed to be central to the degradation of collagen II and proteoglycan seen in arthritis. A number of inhibitors of various of these enzymes are under pre-clinical or clinical investigation.

A number of broadly immunosuppressive drugs are in clinical testing for use in arthritis, including cyclosporine A and mycophenolate mofetil. As a wide range of cytokines are found in arthritic joints, anti-arthritis therapies have targeted cytokine pathways including those of IL-1, IL-2, IL-4, IL-10, IL-11, TGF.beta., and TNF.alpha., as well as, chemokine pathways (Feldmann et al., 1997). In particular, proinflammatory pathways of IL-1 have been targeted both by attack of IL-1 directly and via the naturally occurring interleukin-1 receptor antagonist molecule.

Methods of administering drug therapy for RA have included, and have been proposed to include, systemic or local delivery of a therapeutic drug and, in the case of proposed gene therapies, of a therapeutic gene. To date, such treatments have fallen short of delivering effective, safe therapy for arthritis for a variety of reasons, including: systemic side effects of many drugs, rapid clearance of therapeutic molecules from injected joints and/or circulation, inefficiency in DNA integration and expression from the genome, limited target cell population associated with some viral delivery vectors, transient gene expression associated with viral vectors which do not readily integrate and induction of an immune response associated with the gene delivery virus.

Use of TNF antagonists, such as soluble TNFRs and anti-TNF antibodies, has shown that a blockade of TNF can reverse effects attributed to TNF including decreases in IL-1, GM-CSF, IL-6, IL-8, adhesion molecules and tissue destruction (Feldmann et al., 1997). Such pleiotropic effects apparently due to the blockade of TNF alone suggests that TNF may lie near the top of the cascade of cytokine mediated events. Elevated levels of TNF-.alpha. are found in the synovial fluid of RA patients (Camussi and Lupia, 1998, Drugs 55:613-620).

The effect of TNF blockade utilizing a hamster anti-mouse TNF antibody was tested in a model of collagen type II arthritis in DBA/1 mice (Williams et al., 1992, Proc. Natl. Acad. Sci. USA, 89:9784-9788). Treatment initiated after the onset of disease resulted in improvement in footpad swelling, clinical score, and histopathology of joint destruction. Other studies have obtained similar results using either antibodies (Thorbecke et al., 1992, Proc. Natl. Acad. Sci. USA, 89:7375-7379) or TNFR constructs (Husby et al., 1988, J. Autoimmun. 1:363-71; Tetta et al., 1990, Ann. Rheum. Dis. 49:665-667; Wooley et al., 1993, J. Immunol. 151:6602-6607; Piguet et al., 1992, Immunology 77:510-514).

Similar results have also been obtained in other animal models of ongoing arthritis. In the rabbit, anti-TNF.alpha. antibody was shown to have an anti-arthritic effect on antigen induced arthritis (Lewthwaite et al., 1995, Ann. Rheum. Dis. 54:366-374). In the rat, anti-TNF therapy has been demonstrated to be effective in adjuvant (Mycobacterium) arthritis (Issekutz et al., 1994, Clin. Exp. Immunol. 97:26-32), in streptococcal cell wall induced arthritis (Schimmer et al., 1997, J. Immunol. 159:4103-4108) and in collagen induced arthritis (Le et al., 1997, Arthritis Rheum. 40:1662-1669).

In the studies described above, the TNF blockade was achieved by systemic delivery of the blocking agent. In a rat collagen arthritis model, delivery of a TNFR gene using an adenoviral vector resulted in transient production of serum levels of TNFR (up to 8 days) and a significant decrease in disease progression when the adenovirus was given to animals undergoing active arthritis (Le et al., 1997). Attempts to deliver the gene directly to the joint were unsuccessful, however, and resulted in an inflammatory reaction to the adenovirus.

A monoclonal antibody directed against TNF.alpha. (infliximab, REMICADE, Centocor), administered with and without methotrexate, has demonstrated clinical efficacy in the treatment of RA (Elliott et al., 1993, Arthritis Rheum. 36:1681-1690; Elliott et al., 1994, Lancet 344:1105-1110). These data demonstrate significant reductions in Paulus 20% and 50% criteria at 4, 12 and 26 weeks. This treatment is administered intravenously and the anti-TNF monoclonal antibody disappears from circulation over a period of two months. The duration of efficacy appears to decrease with repeated doses. The patient can generate antibodies against the anti-TNF antibodies which limit the effectiveness and duration of this therapy (Kavanaugh et al., 1998, Rheum. Dis. Clin. North Am. 24:593-614). Administration of methotrexate in combination with infliximab helps prevent the development of anti-infliximab antibodies (Maini et al., 1998, Arthritis Rheum. 41:1552-1563). Infliximab has also demonstrated clinical efficacy in the treatment of the inflammatory bowel disorder Crohn's disease (Baert et al., 1999, Gastroenterology 116:22-28).

Clinical trials of a recombinant version of the soluble human TNFR (p75) linked to the Fc portion of human IgG1 (sTNFR(p75):Fc, ENBREL, Immunex) have shown that its administration resulted in significant and rapid reductions in RA disease activity (Moreland et al., 1997, N. Eng. J. Med., 337:141-147). In addition, preliminary safety data from an ongoing pediatric clinical trial for sTNFR(p75):Fc indicate that this drug is generally well-tolerated by patients with juvenile rheumatoid arthritis (JRA) (Garrison et al, 1998, Am. College of Rheumatology meeting, Nov. 9, 1998, abstract 584).

As noted above, ENBREL is a dimeric fusion protein consisting of the extracellular ligand-binding portion of the human 75 kilodalton (p75) TNFR linked to the Fc portion of human IgG1. The Fc component of ENBREL contains the CH2 domain, the CH3 domain and hinge region, but not the CH1 domain of IgG1. ENBREL is produced in a Chinese hamster ovary (CHO) mammalian cell expression system. It consists of 934 amino acids and has an apparent molecular weight of approximately 150 kilodaltons (Smith et al., 1990, Science 248:1019-1023; Mohler et al., 1993, J. Immunol. 151:1548-1561; U.S. Pat. No. 5,395,760 (Immunex Corporation, Seattle, Wash.); U.S. Pat. No. 5,605,690 (Immunex Corporation, Seattle, Wash.).

Approved by the Food and Drug administration (FDA) (Nov. 2, 1998), ENBREL is currently indicated for reduction in signs and symptoms of moderately to severely active rheumatoid arthritis in patients who have had an inadequate response to one or more disease-modifying antirheumatic drugs (DMARDs). ENBREL can be used in combination with methotrexate in patients who do not respond adequately to methotrexate alone. ENBREL is also indicated for reduction in signs and symptoms of moderately to severely active polyarticular-course juvenile rheumatoid arthritis in patients who have had an inadequate response to one or more DMARDs (May 28, 1999). ENBREL is given to RA patients at 25 mg twice weekly as a subcutaneous injection.

Currently, treatments using the sTNFR(p75):Fc (ENBREL, Immunex) preparations, including those described above, are administered subcutaneously twice weekly, which is costly, unpleasant and inconvenient for the patient. “Important Drug Warning” on World Wide Web at fda.gov/medwatch/safety/1999/enbrel.htm; “New Warning for Arthritis Drug, ENBREL” on World Wide Web at fda.gov/bbs/topics/ANSWERS/ANS00954.html; “ENBREL Injections Difficult for Some Patients” at dailynews.yahoo.com/h/nm/20000516/hl/arthritis_drugs.sub.-1.html. Further, relief afforded by this treatment is not sustained. Symptoms associated with an arthritic condition are reduced during treatment with sTNFR (p75): Fc but return upon discontinuation of this therapy, generally within one month. Complications have arisen, including local reactions at the site of injection. Moreover, long-term systemic exposure to this TNF-.alpha. antagonist can impose a risk for increased viral and bacterial infections and possibly cancer. Since this product was first introduced, serious infections, some involving death, have been reported in patients using ENBREL. “Product Information” on World Wide Web at enbrel.com/patient/html/patpi.htm; “Proven Tolerability” on World Wide Web at enbrel.com/patient/html/patsafety.htm.

Additional relevant references include: U.S. Pat. Nos. 5,858,775; 5,858,355; 5,858,351; 5,846,528; 5,843,742; 5,792,751; 5,786,211; 5,780,447; 5,766,585; 5,633,145; International Patent publications WO 95/16353; WO 94/20517; WO 92/11359; Schwarz, 1998, Keystone Symp., January 23-29, abstract 412; Song et al. (1998) J. Clin. Invest. 101:2615-2621; Ghivizzani et al., 1998, Proc. Natl. Acad. Sci. USA 95:4613-4618; Kang et al., 1997, Biochemical Society Transactions 25:533-537; Robbins et al., 1997, Drug News & Perspect. 10:283-292; Firestein et al., 1997, N. Eng. J. Med. 337:195-197; Muller-Ladner et al., 1997, J. Immunol. 158:3492-3498; and Pelletier et al., 1997, Arthritis Rheum. 40:1012-1019. TNF-αc has been strongly implicated as a major participant in the inflammatory cascade that leads to the joint damage and destruction of diseases such as rheumatoid arthritis (RA), psoriatic arthritis (PsA) and ankylosing spondylitis (AS). Although there is no cure, treatment has been revolutionized by the advent of anti-TNF-α therapies. These include etanercept (Enbrelα), infliximab (Remicadeα) and adalimumab (Humiraα), which consist of soluble TNF receptors, chimeric human-mouse anti-TNF-α monoclonal antibodies and fully human anti-TNF-α monoclonal antibodies, respectively. Clinical studies have shown these products to improve the sighs and symptoms, inhibit the structural damage, and impact functional outcomes in patients with these inflammatory arthritides (Braun and Sieper, 2004; Criscione and St Clair, 2002; Gardner, 2005).

However, some patients with inflammatory arthritis have one or more persistently symptomatic joints despite systemic TNF-α blockade. The reason some patients do not have a complete response to systemic anti-TNF-α agents is not clear. The response to anti-TNF-α agents is relative. For example, approximately 60-70% of RA patients achieve an ACR 20, which consists of a reduction of at least 20 percent in the number of both swollen and tender joints and improvement of at least 20 percent in at least three of the following: the patient's assessment of pain, the physician's global assessment of disease status, the patient's assessment of disability, and values for acute phase reactants. Approximately 40% of patients achieve an ACR 50, which is a 50 percent improvement, and only ˜15% of patients achieve an ACR 70, which is a 70 percent improvement (Gardner, 2005). The net effect is that most patients still have significant room for improvement in inflammation and tender and swollen joint counts.

Etanercept has been administered directly into the joints of a limited number of patients with inflammatory arthritis (Arnold et al., 2003; Bliddal et al., 2002; Osborn, 2002). In a small, double-blind, placebo-controlled study, improvement in joint swelling, tenderness and range of motion was noted in 10 subjects with RA who received an intra-articular injection of 12.5 mg etanercept compared to 10 subjects who received placebo (Osborn, 2002). In a small, dose-escalation study of intra-articular injection of increasing doses of etanercept, improvement in synovitis was noted in RA patients who received the highest dose (8 mg) (Bliddal et al., 2002). In general, intra-articular administration of etanercept was well-tolerated, but joints would likely need to be injected frequently, because of the short half-life of the protein.

There is a need for new, effective forms of treatment for arthritic disorders such as RA, PsA AS, or osteoarthritis wherein one or more joints remains persistently symptomatic despite administration of systemic polypeptide proinflammatory cytokine antagonists, particularly treatments that can provide sustained, controlled therapy for one or more persistently symptomatic joints that do not respond or do not respond completely to systemic polypeptide proinflammatory antagonists. The present invention provides methods for the effective treatment of arthritic inflammatory processes of persistent symptomatic joints of an individual being treated with systemically with pro-inflammatory polypeptide antagonists.

All publications and references cited herein are hereby incorporated by reference in their entirety.

### SUMMARY OF THE INVENTION

The present invention provides methods for treating inflammatory arthritis in an individual, comprising administering to the individual an effective amount of AAV (rAAV) vector comprising a polynucleotide encoding a pro-inflammatory cytokine anatagonist, wherein the individual is being (which includes the individual has been) treated systemically with a polypeptide pro-inflammatory antagonist but still has one or more persistently symptomatic joints.

The present invention also provides methods for treating inflammatory arthritis in an individual, comprising administering to a persistently symptomatic joint of the individual an effective amount of an recombinant AAV (rAAV) vector comprising a polynucleotide encoding a TNF antagonist, wherein the individual is being treated systemically with an art recognized effective amount of a polypeptide TNF-α antagonist but still has one or more persistently symptomatic joints despite the systemic polypeptide TNF-α antagonist treatment.

The present invention also provides methods for enhancing the treatment effect of a polypeptide TNF-α antagonist in an individual, comprising administering to a persistently symptomatic joint of the individual an effective amount of a recombinant AAV (rAAV) vector comprising a polynucleotide encoding a TNF antagonist, wherein the individual is being treated systemically with a polypeptide TNF-α antagonist but still has one or more persistently symptomatic joints despite the systematic polypeptide TNF-α antagonist treatment.

The individual is a mammal, including human, horse, dog, cat, and cow.

In some embodiments, the methods employ administering a rAAV vector to deliver a polynucleotide encoding a TNF-α antagonist to the individual in conjunction with systemic delivery of a polypeptide TNF-α antagonist. In some embodiments, the rAAV vector is administered locally or regionally to a joint. In some embodiments, the rAAV vector is administered by intra-articular injection. In some embodiments, the rAAV vector is administered to the individual at a dosage between about 1×10¹¹ to about 1×10¹² DRP/ml of joint volume, between 1×10¹² to 1×10¹³ DRP/ml of joint volume, or between 1×10¹³ to 1×10¹⁴ DRP/ml of joint volume. In some embodiments, the rAAV vector is administered to the individual every six weeks, eight weeks, twelve weeks, sixteen weeks, 20 weeks, up to or about six months, or a year. In some embodiments the target joint is a hip, knee, ankle, wrist, metacarpal, or spinal joint. In some embodiments the rAAV vector is administered to a single target joint, two target joints, three target joints, four target joints, up to a plurality of target joints.

In some embodiments, the TNF antagonist encoded by the polynucleotide is a TNF-α antagonist. In some embodiments, the rAAV vector comprises a polynucleotide encoding a soluble tumor necrosis factor receptor (TNFR). In some embodiments, the rAAV vector comprises a polynucleotide encoding a p75 TNFR polypeptide. In some embodiments, the rAAV vector comprises a polynucleotide encoding an Fc (constant domain of an immunoglobulin molecule):p75 fusion polypeptide. In some embodiments, the rAAV vector comprises a polynucleotide encoding a fusion polypeptide in which the extracellular domain of TNFR is fused to Fc.

In some embodiments, the rAAV vector of the invention further comprise a polynucleotide encoding an IL-1 antagonist, such as an IL-1 receptor type II polypeptide.

In some embodiments, the systemic polypeptide treatment includes treatment with etanercept (Enbrelα®), infliximab (Remicadeα®), adalimumab (Humiraα®) and Anakinra. These polypeptides are soluble TNF receptors, chimeric human-mouse anti-TNF-α monoclonal antibodies, fully human anti-TNF-α monoclonal antibodies, and a soluble IL-1 receptor respectively.

In some embodiments, the systemic polypeptide treatment includes treatment with any polypeptide that blocks the TNF, and/or other proinflammatory cytokine pathways, such as those of IL-1, IL-2, IL-4, IL-10, IL-11, TGF.beta., and TNF.alpha., as well as, chemokine pathways (Feldmann et al., 1997). Proinflammatory pathways of IL-1 have been targeted both by attack of IL-1 directly and via the naturally occurring interleukin-1 receptor antagonist molecule or other IL-1 antagonists, such as an IL-1 receptor type II polypeptide.

The invention also provides use of the rAAV vector described herein for use in any of the methods described herein, or for the manufacture of a medicament for use in any of the methods described herein; for example, for treating inflammatory arthritis.

### BRIEF DESCRIPTION OF THE FIGURES

FIG. 1 depicts the amino acid sequence of a TNFR:Fc fusion polypeptide (SEQ ID NO:1) from U.S. Pat. No. 5,605,690.

FIGS. 2A and 2B depicts the polynucleotide (SEQ ID NO:2) and amino acid sequences (SEQ ID NO:1) of a TNFR:Fc fusion polypeptide from U.S. Pat. No. 5,605,690.

FIG. 3 depicts the rAAV vector containing the TNFR:Fc fusion polypeptide.

FIG. 4 depicts the amino acid (SEQ ID NO:3) and polynucleotide (SEQ ID NO:4) sequences of a human IL-1R type II from GenBank U74649.

FIG. 5 is a graph depicting the grouped aggregate clinical data of individuals depicting the change from baseline in the tenderness and swelling index of target joints treated with the rAAV vector containing the polynucleotide encoding TNFR:Fc either alone or in conjunction with systemic TNFα antagonists at weeks 1, 4, and 12.

FIG. 6 is a graph depicting percent change from baseline based on the grouped aggregated clinical data. The individuals in each group (9 individuals for placebo, 8 individuals being treated with 1×10¹¹ DRP/ml rAAV, or 10 individuals being treated with 1×10¹² DRP/ml rAAV) were also concurrently treated with a polypeptide TNF-alpha antagonist. Each bar in the graph represents percentage change from baseline in the tenderness and swelling index of target joints in the treated group.

### DETAILED DESCRIPTION

We have discovered compositions and methods for reducing or lowering levels of TNF in target joint and for palliating TNF-associated disorders of an individual with persistent symptomatic joints or tissues despite treatment with polypeptide TNF antagonist therapy Included are methods for reducing inflammatory responses in a subject by reducing levels of TNF activity by the administration of a combination of a systemic polypeptide TNF antagonist and an intra-articular administration of a rAAV vector containing a polynucleotide encoding a TNF antagonist.

The invention described herein provides materials and methods for use in the delivery to and expression of a polynucleotide encoding a TNF antagonist in an individual that is being treated systemically with a polypeptide TNF antagonist. The polynucleotide encoding a TNF antagonist is delivered (e.g., via intra-articular injection) to the persistently symptomatic joint of the individual through a recombinant adeno-associated virus (rAAV) vector, a vector which integrates into the genome of the host cell. Introduction of rAAV DNA into cells generally leads to long-term persistence and expression of DNA without disturbing the normal metabolism of the cell. The polypeptide antagonist is delivered systemically to the mammal via standard techniques known in the art including direct intramuscular injection, intraperitoneal injection, intravenous, intra-articular, subcutaneously, or intradermally. Thus, the invention provides a continuous source of a TNF antagonist polypeptide encoded from the rAAV vectors of the invention loco-regionally to the persistently symptomatic joint as well systemically via the systemic polypeptide agent administered to the individual. This is a distinct and significant advantage for persistently symptomatic joints over previously described treatment modalities (i.e., exogenous administration of polypeptide therapeutic agents alone), which confer only transient benefits.

### DEFINITIONS

As used in the specification and claims, the singular form “a”, “an” and “the” include plural references unless the context clearly dictates otherwise. For example, the term “a cell” includes a plurality of cells, including mixtures thereof.

A “TNF antagonist” as used herein refers to a polypeptide that binds TNF and inhibits and/or hinders TNF activity as reflected in TNF binding to a TNF-receptor including any of the following: (a) TNFR, preferably endogenous (i.e., native to the individual or host), cell membrane bound TNFR; (b) the extracellular domain(s) of TNFR; and/or (c) the TNF binding domains of TNFR (which may be a portion of the extracellular domain). TNF antagonists include, but are not limited to, TNF receptors (or appropriate portions thereof, as described herein) and anti-TNF antibodies. As used herein, the “biological activity” of a TNF antagonist is to bind to TNF and inhibit and/or hinder TNF from binding to any of the following: (a) TNFR, preferably endogenous, cell membrane bound TNFR; (b) the extracellular domain(s) of TNFR; and (c) the TNF binding domains of TNFR (which may be a portion of the extracellular domain). A TNF antagonist can be shown to exhibit biological activity using assays known in the art to measure TNF activity and its inhibition, an example of which is provided herein.

“TNF-associated disorders” are those disorders or diseases that are associated with, result from, and/or occur in response to, elevated levels of TNF. Such disorders may be associated with episodic or chronic elevated levels of TNF activity and/or with local or systemic increases in TNF activity. Such disorders include, but are not limited to, inflammatory diseases, such as arthritis.

A “pro-inflammatory antagonist” as used herein refers to a polypeptide that binds an inflammatory cytokine and inhibits and/or hinders the activity of the inflammatory cytokines as reflected in the inhibition of binding of the proinflammatory cytokine binding to its cytokine-receptor. Examples of proinflammatory cytokines include but are not limited to IFN y, IL-6, IL-2, IL4, IL-10, IL13, and IL4. TNFα, and IL-1 are also considered pro-inflammatory cytokines.

A “Persistently Symptomatic Joint (s)” as used herein refers to a joint(s) that exhibits tenderness, swelling, pain, or decreased mobility such that the individual's (1) quality of life is negatively impacted; and/or (2) performance of daily activities is inhibited; and/or (3) is functionally impaired despite the individual receiving systemic polypeptide pro-inflammatory antagonists at doses recognized in the art as effective.

A “Target Joint” as used herein refers to a persistently symptomatic joint that has been administered a rAAV vector containing a polynucleotide encoding a pro-inflammatory antagonist. The rAAV vector of the invention includes an rAAV containing a polynucleotide encoding TNFr:Fc.

As used herein, the terms “TNF receptor polypeptide” and “TNFR polypeptide” refer to polypeptides derived from TNFR (from any species) which are capable of binding TNF. Two distinct cell-surface TNFRs have described: Type II TNFR (or p75 TNFR or TNFRII) and Type I TNFR (or p55 TNFR or TNFRI). The mature full-length human p75 TNFR is a glycoprotein having a molecular weight of about 75-80 kilodaltons (kD). The mature full-length human p55 TNFR is a glycoprotein having a molecular weight of about 55-60 kD. The preferred TNFR polypeptides of this invention are derived from TNFR Type I and/or TNFR type II.

TNFR polypeptides, such as “TNFR”, “TNFR:Fc” and the like, when discussed in the context of the present invention and compositions therefor, refer to the respective intact polypeptide (such as, TNFR intact), or any fragment or derivative thereof (such as, an amino acid sequence derivative), that exhibits the desired biological activity (i.e., binding to TNF). A “TNFR polynucleotide” is any polynucleotide which encodes a TNFR polypeptide (such as a TNFR:Fc polypeptide).

As used herein, an “extracellular domain” of TNFR refers to a portion of TNFR that is found between the amino-terminus of TNFR and the amino-terminal end of the TNFR transmembrane region. The extracellular domain of TNFR binds TNF.

A “IL-1 antagonist” as used herein refers to a polypeptide that binds interleukin I (IL-1) and inhibits and/or hinders IL-1 activity as reflected in IL-1 binding to an IL-1 receptor including any of the following: (a) IL-1 receptor (IL-1R), preferably endogenous (i.e., native to the individual or host), cell membrane bound IL-1R (b) the extracellular domain(s) of IL-1R; and/or (c) the IL-1 binding domains of IL-1R (which may be a portion of the extracellular domain). IL-1 antagonists include, but are not limited to, IL-1 receptors (or appropriate portions thereof, as described herein) and anti-IL-1 antibodies. As used herein, the “biological activity” of an IL-1 antagonist is to bind to IL-1 and inhibit and/or hinder IL-1 from binding to any of the following: (a) IL-1R, preferably endogenous, cell membrane bound IL-1R; (b) the extracellular domain(s) of IL-1R; and/or (c) the IL-1 binding domains of IL-1R (which may be a portion of the extracellular domain). An IL-1 antagonist can be shown to exhibit biological activity using assays known in the art, including IL-1 inhibition assays, which are described herein as well as in the art.

As used herein, the term “IL-1 receptor polypeptide” refers to polypeptides derived from IL-1 receptor (from any species) which are capable of binding IL-1. IL-1R polypeptides, when discussed in the context of the present invention and compositions therefore, refer to the respective intact polypeptide (such as intact IL-1R), or any fragment or derivative thereof (such as, an amino acid sequence derivative), that exhibits the desired biological activity (i.e., binding to IL-1). A “IL-1R polynucleotide” is any polynucleotide which encodes a IL-1R polypeptide.

As used herein, an “extracellular domain” of IL-1R refers to a portion of IL-1R that is found between the amino-terminus of IL-1R and the amino-terminal end of the IL-1R transmembrane region. The extracellular domain of IL-1R binds IL-1.

The terms “polypeptide”, “peptide” and “protein” are used interchangeably herein to refer to polymers of amino acids of any length. The terms also encompass an amino acid polymer that has been modified; for example, disulfide bond formation, glycosylation, lipidation, or conjugation with a labeling component.

A “chimeric polypeptide” or “fusion polypeptide” is a polypeptide comprising regions in a different position than occurs in nature. The regions may normally exist in separate proteins and are brought together in the chimeric or fusion polypeptide, or they may normally exist in the same protein but are placed in a new arrangement in the chimeric or fusion polypeptide. A chimeric or fusion polypeptide may also arise from polymeric forms, whether linear or branched, of TNFR polypeptide(s).

The terms “polynucleotide” and “nucleic acid”, used interchangeably herein, refer to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

A “chimeric polynucleotide” or “fusion polynucleotide” is a polynucleotide comprising regions in a different position than occurs in nature. The regions may normally exist in separate genes and are brought together in the chimeric or fusion polynucleotide, or they may normally exist in the same gene but are placed in a new arrangement in the chimeric or fusion polynucleotide.

“AAV” is an abbreviation for adeno-associated virus, and may be used to refer to the virus itself or derivatives thereof. The term covers all subtypes and both naturally occurring and recombinant forms, except where required otherwise.

An “rAAV vector” as used herein refers to an AAV vector comprising a polynucleotide sequence not of AAV origin (i.e., a polynucleotide heterologous to AAV), typically a sequence of interest for the genetic transformation of a cell. The heterologous polynucleotide is flanked by at least one, preferably two, AAV inverted terminal repeat sequences (ITRs). As described herein, an rAAV vector can be in any of a number of forms, including, but not limited to, plasmids, linear artificial chromosomes, complexed with lipids, encapsulated within liposomes and, most preferably, encapsidated in a viral particle, particularly an AAV.

An “rAAV virus” or “rAAV viral particle” refers to a viral particle composed of at least one AAV capsid protein (preferably by all of the capsid proteins of a wild-type AAV) and an encapsidated rAAV.

A “gene” refers to a polynucleotide containing at least one open reading frame that is capable of encoding a particular protein after being transcribed and translated. 100671 “Recombinant”, as applied to a polynucleotide means that the polynucleotide is the product of various combinations of cloning, restriction or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A cell is said to be “stably” altered, transduced, or transformed with a genetic sequence if the sequence is available to perform its function during extended culture of the cell in vitro. In preferred examples, such a cell is “inheritably” altered in that a genetic alteration is introduced which is also inheritable by progeny of the altered cell.

A “host cell” includes an individual cell or cell culture which can be or has been a recipient for vector(s) or for incorporation of polynucleotides and/or proteins. Host cells include progeny of a single host cell, and the progeny may not necessarily be completely identical (in morphology or in genomic of total DNA complement) to the original parent cell due to natural, accidental, or deliberate mutation. A host cell includes cells transfected in vivo with a polynucleotide(s) of this invention.

An “individual” (alternatively referred to as a “subject”) is a mammal, more preferably a human. Mammals also include, but are not limited to, farm animals (such as cows), sport animals, pets (such as cats, dogs, horses), primates, mice and rats.

An “effective amount” is an amount sufficient to effect or achieve a beneficial or desired clinical result. An effective amount can be administered in one or more administrations. For purposes of this invention, an “effective amount” is an amount that achieves any of the following: reduction of TNF levels; reduction of an inflammatory response; and/or palliation, amelioration, stabilization, reversal, slowing or delay in the progression or a sign or symptom of the disease state including a reduction in tenderness and/or swelling in a target joint.

As used herein, “in conjunction with”, “concurrent”, or “concurrently”, as used interchangeably herein, refers to administration of one treatment modality in addition to another treatment modality, such as systemic administration of a polypeptide TNF antagonist to an individual in addition to the delivery of an rAAV containing a polynucleotide encoding a TNF antagonist to a target joint of the same individual. As such, “in conjunction with” refers to administration of one treatment modality before, during or after delivery of the other treatment modality to the subject.

“ACR 20” is a term well understood in the art and refers to a score that is defined by the American College of Rheumatology based on at least a 20% reduction in the number of swollen and tender joints and improvement of at least 20% in at least three of the following: the patient's assessment of pain, the physician's global assessment of disease status, the patient's global assessment of disease status, the patient's assessment of disability, and values for acute phase reactants (either the erythrocyte sedimentation rate or the level of C reactive protein) (Felson et al., 1995).

An “arthritic condition” or “arthritic syndrome” is a term well-understood in the art and refers to a state characterized by inflammation of a joint or joints.

As used herein, “enhanced” refers to an improved beneficial or desired clinical result obtained in a persistently symptomatic joint including the target joint injected (e.g., loco-regionally or intra-articularly) with the rAAV vector containing the polynucleotide encoding the pro-inflammatory antagonist in combination with systemic polypeptide proinflammatory antagonist therapy compared to the clinical result obtained for the joint receiving only systemic polypeptide proinflammatory antagonist therapy. For purposes of this invention, improved beneficial or desired clinical results include, but are not limited to, a greater alleviation of signs or symptoms of inflammation, increase diminishment of extent of disease, stabilized (i.e., not worsening) state of disease, preventing spread of disease, delaying or slowing of disease progression, amelioration or palliation of the disease state, and remission (whether partial or total), whether detectable or undetectable. Examples of “enhanced” treatment effects” or “enhanced therapeutic effect include a reduction in tenderness and or swelling of the target joint, increased mobility of the target joint, increased functioning of the target joint, and/or an improved quality of life of the individual receiving the method of the invention. “Enhanced” can also mean a prolonged time to intra-articular re-administration of the rAAV vector containing the polynucleotide encoding the TNF antagonist based on a sustained treatment effect.

As used herein “polypeptide TNF antagonists”, and “polypeptide TNF-α antagonists” used in systemic polypeptide TNF antagonist treatment refer to polypeptide or protein based biologics which act to block the TNF cascades or other cytokines of the proinflammatory cascade associated with arthritic disorders or syndromes. The term also includes any chemical modifications, alterations (including amino acid substitutions), synthetic and natural variants, or biologically engineered variants, which act to block the TNF cascades or other cytokines of the pro-inflammatory cascade, and further include including antibodies directed to receptors of the pro-inflammatory cascade or TNF cascade associated with TNF associated disorders. “TNF polypeptide antagonists” include etanercept (Enbrelα®), infliximab (Remicadeα®) and adalimumab (Humiraα®), which consist of soluble TNF receptors, chimeric human-mouse anti-TNF-α monoclonal antibodies and fully human anti-TNF-α monoclonal antibodies, respectively.

As used herein, “treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment. For example, treatment of an individual may be undertaken to decrease or limit the pathology associated with elevated levels of TNF.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides. The term “biological sample” encompasses a clinical sample, and also includes cells in culture, cell supernatants, cell lysates, serum, plasma, biological fluid, and tissue samples.

“Palliating” a disease means that the extent and/or undesirable clinical manifestations of a disease state are lessened and/or time course of the progression is slowed or lengthened, as compared to not administering rAAV vectors of the present invention.

### General Techniques

The practice of the present invention will employ, unless otherwise indicated, conventional techniques of molecular biology, virology, animal cell culture and biochemistry which are within the skill of the art. Such techniques are explained fully in the literature. See, for example, “Molecular Cloning: A Laboratory Manual”, Second Edition (Sambrook, Fritsch & Maniatis, 1989); “Animal Cell Culture” (R. I. Freshney, ed., 1987); “Gene Transfer Vectors for Mammalian Cells” (J. M. Miller & M. P. Calos, eds., 1987); “Current Protocols in Molecular Biology” (F. M. Ausubel et al., eds., 1987); “Current Protocols in Protein Science” (John E Coligan, et al. eds. Wiley and Sons, 1995); and “Protein Purification: Principles and Practice” (Robert K. Scopes, Springer-Verlag, 1994).

rAAV Vectors for Delivery of polynucleotide TNF Antagonists

This invention provides a method for administration of recombinant AAV (rAAV) vectors containing a polynucleotide encoding a TNF antagonist to persistently symptomatic joints of an individual receiving an art recognized effective amount of a polypeptide TNF antagonist in an amount sufficient to produce an enhanced treatment effect of the target joint. Generally, these rAAV vectors comprise a polynucleotide encoding a TNF antagonist. The TNF antagonist is secreted by the cell that receives the rAAV vector; preferably the TNF antagonist is soluble (i.e., not attached to the cell). For example, soluble TNF antagonists are devoid of a transmembrane region and are secreted from the cell. Techniques to identify and remove polynucleotide sequences which encode transmembrane domains are known in the art. Preferably the TNF antagonist is a TNFR, or a TNFR polypeptide (including biologically active derivative(s) thereof). In the present invention, a preferred TNFR is derived from the p75 TNFR.

The rAAV vectors that can be administered according to the present invention also include rAAV vectors comprising a polynucleotide which encodes a RNA (e.g., RNAi) that inhibits the generation of a pro-inflammatory cytokine (e.g., a TNF).

A rAAV vector of this invention comprises a heterologous (i.e. non-AAV) polynucleotide of interest in place of the AAV rep and/or cap genes that normally make up the bulk of the AAV genome. As in the wild-type AAV genome, however, the heterologous polynucleotide is preferably flanked by at least one, more preferably two, AAV inverted terminal repeats (ITRs). Variations in which a rAAV construct is flanked by a only a single (typically modified) ITR have been described in the art and can be employed in connection with the present invention.

### TNF Polypeptide Antagonists

In the present invention, a polypeptide TNF antagonist is supplied to an individual, preferably a mammal, most preferably a human, as an expressed product of a polynucleotide which encodes a TNF antagonist. As defined, such a TNF antagonist may be any polypeptide which binds to TNF including, but not limited to, a TNFR polypeptide and an anti-TNF antibody.

Preferably, the TNF antagonist is a TNFR polypeptide. TNFR polypeptide may be an intact TNFR (preferably from the same species that receives the rAAV) or a suitable fragment of TNFR. U.S. Pat. No. 5,605,690 provides examples of TNFR polypeptides, including soluble TNFR polypeptides, appropriate for use in the present invention. Preferably, the TNFR polypeptide comprises an extracelluar domain of TNFR. More preferably, the TNFR polypeptide is a fusion polypeptide comprising an extracellular domain of TNFR linked to a constant domain of an immunoglobulin molecule; still more preferably, the TNFR polypeptide is a fusion polypeptide comprising an extracellular domain of the p75 TNFR linked to a constant domain of an IgG1 molecule. Preferably when administration to humans is contemplated, an Ig used for fusion proteins is human, preferably human IgG1.

Monovalent and multivalent forms of TNFR polypeptides may be used in the present invention. Multivalent forms of TNFR polypeptides possess more than one TNF binding site. Multivalent forms of TNFR polypeptides may be encoded in an rAAV vector, for example, through the repeated ligation of polynucleotides encoding TNF binding domains, each repeat being separated by a linker region. Preferably, the TNFR of the present invention is a bivalent, or dimeric, form of TNFR. For example, as described in U.S. Pat. No. 5,605,690 and in Mohler et al., 1993, J. Immunol., 151:1548-1561, a chimeric antibody polypeptide with TNFR extracellular domains substituted for the variable domains of either or both of the immunoglobulin heavy or light chains would provide a TNFR polypeptide for the present invention. Generally, when such a chimeric TNFR:antibody polypeptide is produced by cells, it forms a bivalent molecule through disulfide linkages between the immunoglobulin domains. Such a chimeric TNFR:antibody polypeptide is referred to as TNFR:Fc.

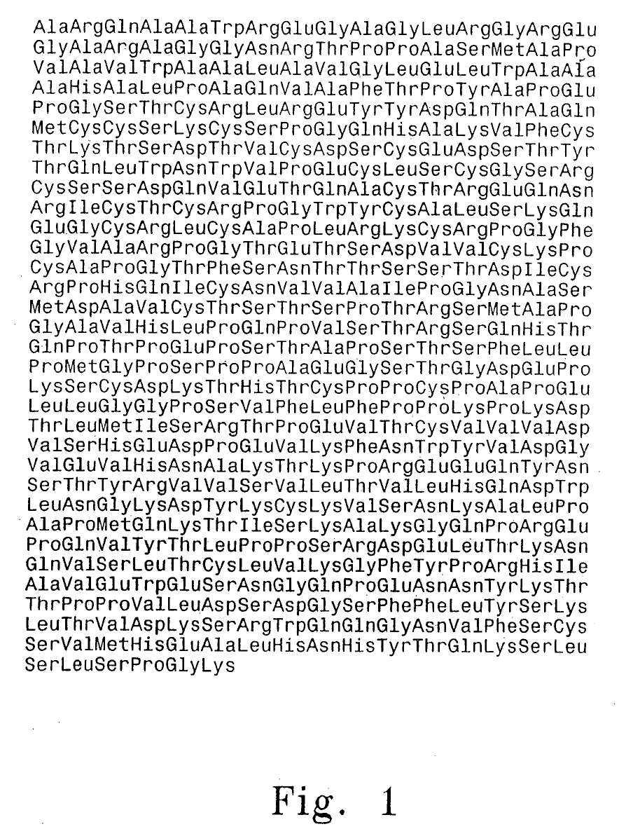

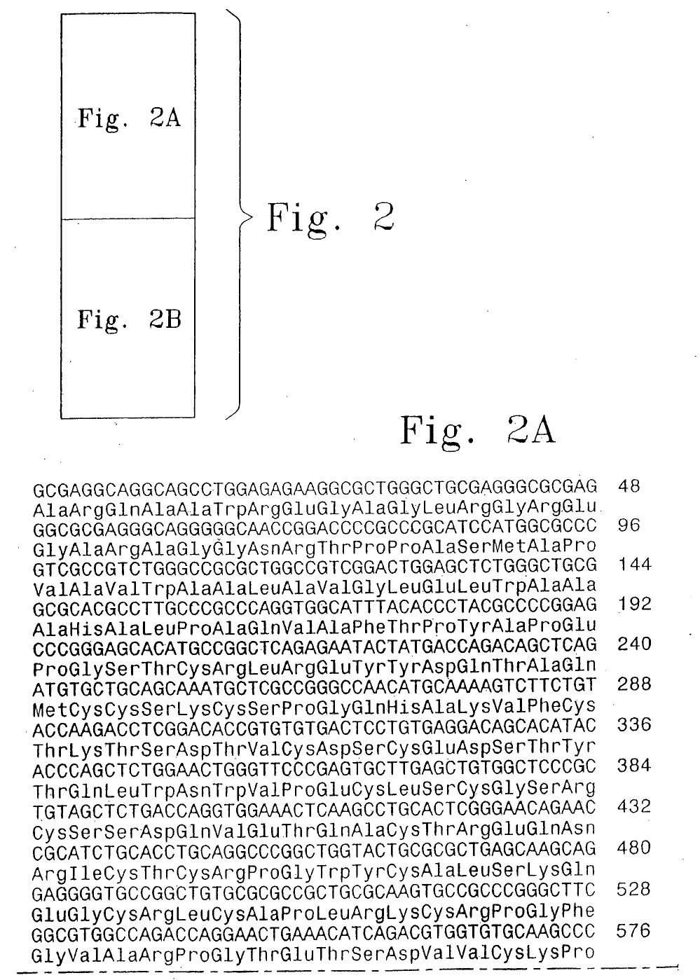

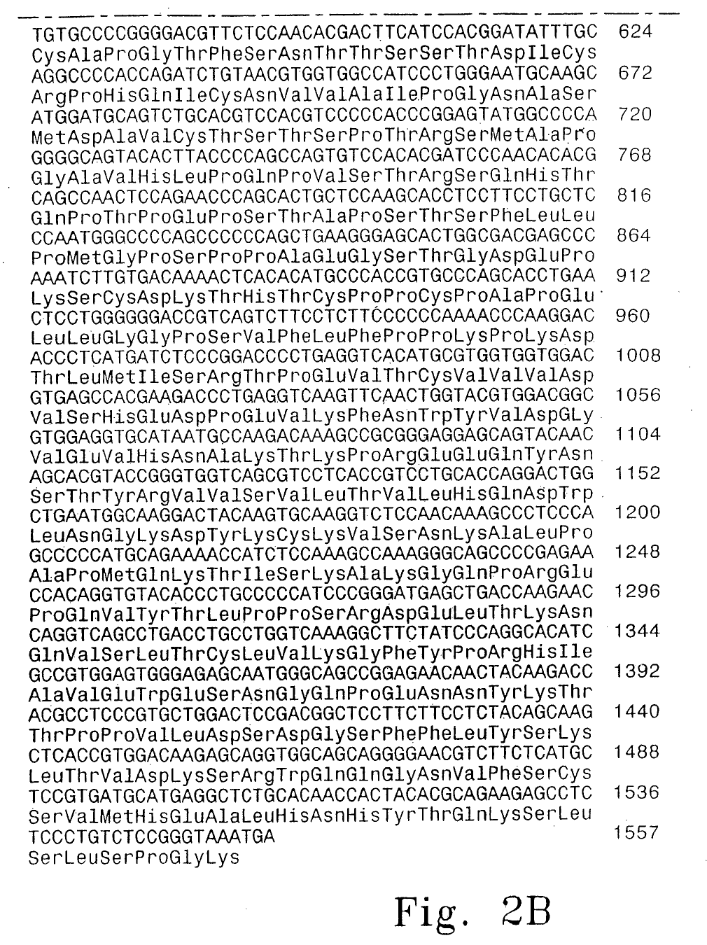

The TNFR polypeptide construct sTNFR(p75):Fc is a preferred embodiment of a TNF antagonist of the present invention. The polypeptide sequence of sTNFR(p75):Fc is depicted in FIG. 1. The coding sequence for this TNF antagonist is found in plasmid pCAVDHFRhuTNFRFc as described in U.S. Pat. No. 5,605,690. Any polynucleotide which encodes this sTNFR(p75):Fc polypeptide is suitable for use in the present invention. A polynucleotide sequence encoding sTNFR(p75):Fc is depicted in FIGS. 2A and 2B.

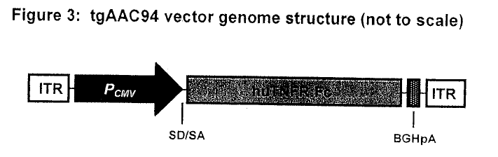

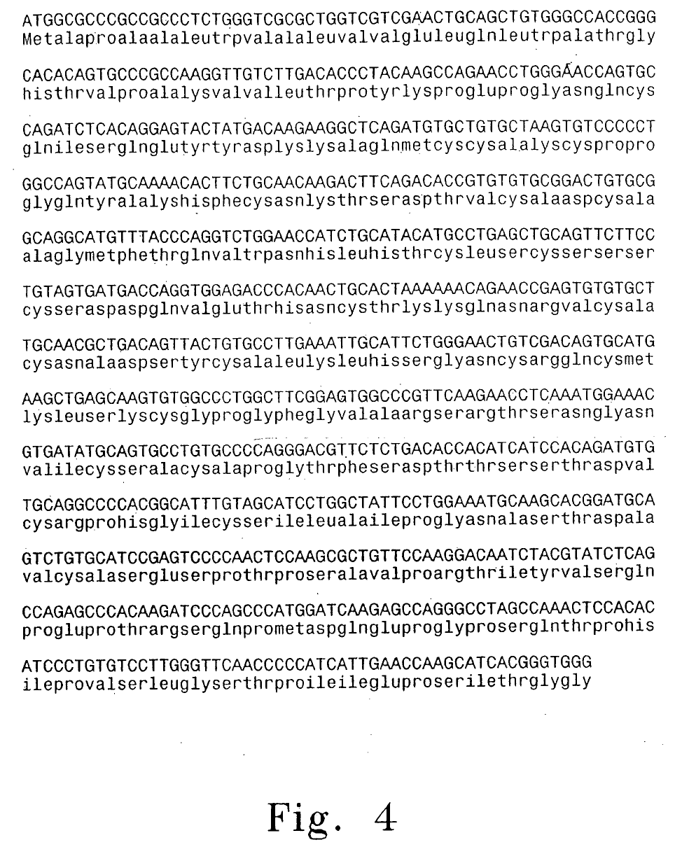

In the present invention, additional TNFR polypeptide sequences include, but are not limited to, those indicated in FIGS. 2 and 3 of U.S. Pat. No. 5,395,760.

Polynucleotides which encode TNFR polypeptides can be generated using methods known in the art from TNFR polynucleotide sequences known in the art. In the present invention, preferable polynucleotide sequences which encode TNFR polypeptides include, but are not limited to, TNFR polynucleotide sequences found in U.S. Pat. Nos. 5,395,760 and 5,605,690 and GenBank entries M32315 (human TNFR) and M59378 (murine TNFRI). Suitable polynucleotides for use in the present invention can be synthesized using standard synthesis and recombinant methods.

Methods to assess TNF antagonist activity are known in the art and exemplified herein. For example, TNF antagonist activity may be assessed with a cell-based competitive binding assay. In such an assay, radiolabelled TNF is mixed with serially diluted TNF antagonist and cells expressing cell membrane bound TNFR. Portions of the suspension are centrifuged to separate free and bound TNF and the amount of radioactivity in the free and bound fractions determined. TNF antagonist activity is assessed by inhibition of TNF binding to the cells in the presence of the TNF antagonist.

As another example, TNF antagonists may be analyzed for the ability to neutralize TNF activity in vitro in a bioassay using cells susceptible to the cytotoxic activity of TNF as target cells, such as L929 cells (see, for example, Example 3). In such an assay, target cells, cultured with TNF, are treated with varying amounts of TNF antagonist and subsequently are examined for cytolysis. TNF antagonist activity is assessed by a decrease in TNF-induced target cell cytolysis in the presence of the TNF antagonist.

The invention also provides a method for administration of recombinant AAV (rAAV) vectors containing a polynucleotide encoding an interleukin 1 (IL-1) antagonist to persistently symptomatic joints of an individual receiving an art recognized effective dose of a polypeptide IL-1 antagonist in an amount sufficient to produce an enhanced treatment effect of the target joint. The cytokine IL-1 has been implicated as a pivotal mediator in both the early and late disease stages of RA (Joosten et al., 1996, Arthritis Rheum. 39:797-809). In RA, IL-1 appears to be involved in infiltration of inflammatory cells and cartilage destruction in the affected joint. A clinical trial with an IL-1 antagonist in patients with RA indicated that blocking IL-1 activity may result in amelioration of RA symptoms (Campion et al., 1996, Arthritis Rheum. 39:1092-1101; Bresnihan et al., 1996, Arthritis Rheum. 39:S73). In a murine arthritis model, a combined anti-TNF.alpha./anti-IL-1 treatment led to both diminished inflammation and to diminished joint cartilage damage (Kuiper et al., 1998, Cytokine 10:690-702).

As IL-1 and TNF appear to mediate different aspects of RA, the present invention provides rAAV vectors comprising a polynucleotide encoding a TNF antagonist (such as sTNFR(p75):Fc) and an IL-1 antagonist (or, the rAAV vector comprises a polynucleotide which encodes a TNF antagonist and an IL-1 antagonist). The present invention also provides rAAV vectors comprising a polynucleotide encoding an IL-1 antagonist. Preferably, the IL-1 antagonist is an IL-1 receptor (IL-1R), or an IL-1R polypeptide (including biologically active derivatives(s) thereof), that exhibits the desired biological activity (i.e., binding to IL-1). Preferably, the IL-1R is derived from IL-1R type II. In the present invention, preferable IL-1R polypeptide sequences include, but are not limited to, that depicted in FIG. 3 and those found in IL-1R GenBank entry U74649 and U.S. Pat. No. 5,350,683. Any polynucleotide which encodes an IL-1R polypeptide is suitable for use in the present invention. A polynucleotide sequence encoding a preferred IL-1R polypeptide is depicted in FIG. 3. Suitable polynucleotides for use in the present invention can be synthesized using standard synthesis and recombinant methods.

Methods to assess IL-1 antagonist activity are known in the art. For example, IL-1 antagonist activity may be assessed with a cell-based competitive binding assay as described herein for TNF antagonists. As another example, IL-1 antagonist activity may be assessed for the ability to neutralize IL-1 activity in vitro in a bioassay for IL-1. In such an assay, a cell line (for example, EL-4 NOB-1) is used that produces interleukin 2 (IL-2) in response to treatment with IL-1. This IL-1 responsive cell line is used in combination with a IL-2 sensitive cell line (for example, CTLL-2). Proliferation of the IL-2 sensitive cell line is dependent on the IL-1 responsive cell line producing IL-2 and thus, is used as a measure of Il-1 stimulation of the IL-1 responsive cell line. IL-1 antagonist activity would be assessed by its ability to neutralize IL-1 activity in such a IL-1 bioassay (Gearing et al., 1991, J. Immunol. Methods 99:7-11; Kuiper et al., 1998).

In preferred embodiments, the vector(s) for use in the methods of the invention are encapsidated into an rAAV virus particle. Accordingly, the invention includes an rAAV virus particle (recombinant because it contains a recombinant polynucleotide) comprising any of the vectors described herein. Methods of producing such particles are known in the art and are described in U.S. Pat. No. 6,596,535.

The present invention also provides compositions containing any of the rAAV vectors (and/or rAAV virus particles comprising the rAAV vectors) described herein. These compositions are especially useful in the methods of the invention in individuals who have persistently symptomatic joints despite treatment with an art recognized effective amount of a polypeptide proinflammatory antagonist.

Generally, the compositions of the invention for use in the method of treating a target joint of an individual with a persistently symptomatic joint comprise an effective amount of an rAAV vector encoding a TNF antagonist, preferably in a pharmaceutically acceptable excipient. As is well known in the art, pharmaceutically acceptable excipients are relatively inert substances that facilitate administration of a pharmacologically effective substance and can be supplied as liquid solutions or suspensions, as emulsions, or as solid forms suitable for dissolution or suspension in liquid prior to use. For example, an excipient can give form or consistency, or act as a diluent. Suitable excipients include but are not limited to stabilizing agents, wetting and emulsifying agents, salts for varying osmolarity, encapsulating agents, and buffers. Excipients as well as formulations for parenteral and nonparenteral drug delivery are set forth in Remington's Pharmaceutical Sciences 19th Ed. Mack Publishing (1995).

Generally, these rAAV compositions are formulated for administration by injection. Preferably these rAAV compositions are formulated for administration by intra-articular injection. Accordingly, these compositions are preferably combined with pharmaceutically acceptable vehicles such as saline, Ringer's balanced salt solution (pH 7.4), dextrose solution, and the like. Although not required, pharmaceutical compositions may optionally be supplied in unit dosage form suitable for administration of a precise amount.

The invention also includes any of the above vectors (or compositions comprising the vectors) for use in treatment of persistently symptomatic joints in individuals with TNF-associated disorders. The invention also includes any of the above vectors (or compositions comprising the vectors) for use in enhancing the treatment effect in a target joint.

Preparation of the rAAV of the Invention

The rAAV vectors of this invention may be prepared using standard methods in the art. Adeno-associated viruses of any serotype are suitable, since the various serotypes are functionally and structurally related, even at the genetic level (see, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. M E Bloom, R M Linden, C R Parrish, Eds.) p 5-14, Hudder Arnold, London, UK (2006); and D E Bowles, J E Rabinowitz, R J Samulski “The Genus Dependovirus” (J R Kerr, S F Cotmore. M E Bloom, R M Linden, C R Parrish, Eds.) p 15-23, Hudder Arnold, London, UK (2006).

Methods of Using rAAV of the Invention

The invention also provides methods in which administration of rAAV vectors to target joints described herein is used to reduce levels of TNF in the target joint. Such methods may be particularly beneficial to individuals with a TNF-associated disorders. Disorders suitable for these methods are those associated with elevated levels of TNF and include, but are not limited to, arthritis (including RA), psoriatic arthritis (PsA), ankylosing spondylitis (AS), osteoarthritis and arthritic joint syndromes associated with other inflammatory diseases including inflammatory bowel diseases (including Crohn's disease and ulcerative colitis), asthma and congestive heart failure wherein the individual has persistently symptomatic joint despite receiving an art recognized effective amount of a polypeptide pro-inflammatory antagonist including a TNFα antagonist.

In one embodiment, methods provided herein for reducing levels of TNF include administration (delivery) of rAAV vectors (or compositions comprising the vectors) to target joints as described herein. In another embodiment, rAAV vectors containing a polynucleotide encoding a TNF antagonist are administered to a persistently symptomatic joint in conjunction with administration of a polypeptide TNF antagonist, such as TNFR or an anti-TNF antibody. The polypeptide TNF antagonist, preferably formulated in compositions with physiologically acceptable carriers known in the art including, excipients or diluents, may be administered by suitable techniques including, but not limited to, intra-articular, intraperitoneal or subcutaneous routes by bolus injection, continuous infusion or sustained release from implants. As discussed below, the rAAV containing a polynucleotide encoding in TNF antagonist preferably formulated in compositions with physiologically acceptable carriers known in the art including, excipients or diluents, may be administered by may also be administered suitable techniques including, but not limited to, intra-articular, loco-regionally by bolus injection, continuous infusion or sustained release from implants directly administered to the target joint.

The invention also provides methods in which administration of rAAV vectors described herein (or compositions comprising an rAAV vector(s) is used to reduce an inflammatory response in a target joint of an individual. Preferably, an inflammatory response is reduced in a connective tissue, including, but not limited to, synovium, cartilage, ligament and tendon of a target joint. A preferred anatomical site for reduction of an inflammatory response is a target joint in an individual with arthritis, such as RA, PsA, or AS. It is understood that an inflammatory response is reduced in an individual with a persistently symptomatic joint when compared to an inflammatory response in an individual prior to receiving rAAV containing a polynucleotide encoding a TNF antagonist or when compared to an inflammatory response in an individual that does not receive a rAAV containing a polynucleotide encoding TNF antagonist.

The invention also provides methods in which administration of rAAV vectors described herein (or compositions comprising an rAAV vector(s)) is used to palliate a TNF-associated disorder of a target joint, including inflammatory diseases such as arthritis (i.e., an arthritic condition) occurring in an individual. Preferably, an arthritic condition is palliated in a target joint, preferably connective tissue which includes, but is not limited to, synovium, cartilage, ligament and tendon. It is understood that an arthritic condition of a target joint is palliated when compared to an arthritic condition in an individual with a persistent symptomatic joint prior to receiving a rAAV containing a polynucleotide encoding a TNF antagonist or when compared to an arthritic condition in an individual that does not receive rAAV containing a polynucleotide encoding a TNF antagonist.

In a preferred embodiment, the rAAV vector (or compositions comprising an rAAV vector(s)) containing a polynucleotide encoding a TNF antagonist is delivered to an arthritic target joint of a mammal thus providing a source of the TNF antagonist at the site of inflammation. Even more preferably, the rAAV vector comprises a polynucleotide encoding sTNFR(p75):Fc.

In another preferred embodiment, the rAAV vector(s) (or compositions comprising an rAAV vector(s)) is delivered via intra-articular injection to a target joint of an individual providing a source of the TNF antagonist and a source of IL-1 antagonist at the site of inflammation. Preferably, the rAAV vector comprises a polynucleotide encoding sTNFR(p75):Fc and a polynucleotide encoding IL-1R.

In another preferred embodiment, a source of the TNF antagonist and a source of IL-1 antagonist are delivered to an target joint of an individual through the administration of at least two different rAAV vectors (or compositions comprising at least two different rAAV vectors). Preferably, one of the rAAV vectors comprises a polynucleotide encoding a TNFR and another one of the rAAV vectors comprises a polynucleotide encoding an IL-1R. In these two different rAAV vectors, the heterologous polynucleotides may be operably linked to transcriptional promoters and/or enhancers which are active under similar conditions or to transcriptional promoters and/or enhancers which are active under different conditions, e.g., independently regulated. In various refinements of administration, the two different rAAV vectors (i.e., one comprising a polynucleotide encoding a TNFR and one comprising a polynucleotide encoding IL-1R) may be administered to the mammal at the same time or at different times, at the same or at different frequencies and/or in the same or at differing amounts.

For any of the above methods, it is understood that one or more rAAV vectors may be administered to the target joint. For example, as discussed above, a vector may be administered that encodes a TNF antagonist, such as TNF receptor (most preferably sTNFR(p75):Fc). Alternatively, an additional vector may be administered to the target joint that encodes an IL-1 antagonist, such as an IL-1 receptor polypeptide. Alternatively, a single vector encoding both a TNF antagonist and an IL-1 antagonist may be administered to the target joint. This single vector may have the coding sequences under control of the same or different transcriptional regulatory elements. If more than one vector is used, it is understood that they may be administered at the same or at different times and/or frequencies to the persistently symptomatic joint.

Further, it is understood that, for any of the above methods, in preferred embodiments, the individual receiving rAAV vector(s) have cells which contain the rAAV vector (after administration), and most preferably have cells in which the rAAV vector(s) is integrated into the cellular genome. Stable integration of rAAV is a distinct advantage, as it allows more persistent expression than episomal vectors. Accordingly, in preferred embodiments, cells (i.e., at least one cell) in the individual comprise stably integrated rAAV. Stated alternatively, for any of the above methods, administration of rAAV(s) results in integration of the rAAV(s) into cellular genomes (although, as is understood by those in the art, not all rAAV vectors need be integrated). Methods of determining and/or distinguishing integrated vs. non-integrated forms, such as Southern detection methods, are well known in art.

A preferred mode of administration of the rAAV compositions is through intra-articular injection of the composition. Preferably, the rAAV composition is delivered to the synovium of the affected joint; more preferably, to synovial cells lining the joint space. Administration to the joint can be single or repeated administrations. Repeated administration would be at suitable intervals, such as about any of the following: once a month, once every 6 weeks, once every two months, once every three months, once every four months, once every five months, once very six months, up to once a year. Repeated administrations may also occur at varying intervals.

The volume of the rAAV vector injected depend on the joint selected for injection. A preferred method of determining the volume is based on current clinical practice with intra-articular injections of steroids in patients with inflammatory arthritis but one skilled in the art will recognize that other methods known in the art including volumetric calculations of joint volume based on radiographic techniques can be utilized to determine the volume of the rAAV vector to be injected. Accordingly in preferred embodiments knees are injected with 5 mL, ankles with 2 mL, elbows with 1.5 mL, wrists with 1 mL, and metacarpophalangeal (MCP) joints with 0.5 mL. For other joints one of ordinary skill in the art can determine the correct volume for injection of the joint.

An effective amount of rAAV (preferably in the form of AAV particles) is administered, depending on the objectives of treatment. An effective amount may be given in single or divided doses. Where a low percentage of transduction can achieve a therapeutic effect, then the objective of treatment is generally to meet or exceed this level of transduction. In some instances, this level of transduction can be achieved by transduction of only about 1 to 5% of the target cells, but is more typically about 20% of the cells of the desired tissue type, usually at least about 50%, preferably at least about 80%, more preferably at least about 95%, and even more preferably at least about 99% of the cells of the desired tissue type.

As a guide, the number of rAAV particles administered per injection is generally between about 1×106 and about 1×10¹⁴ particles, preferably, between about 1×107 and 1×1013 particles, more preferably about 1×109 and 1×1012 particles and even more preferably about 1×1011 particles.

The number of rAAV particles administered per joint by intra-articular injection, for example, is generally at least about 1×1011, and is more typically about 5×1011, about 1×1012, and on some occasions about 1×1013 particles, including both DNAse resistant and DNAse susceptible particles. In terms of DNAse resistant particles, the dose is generally be between about 1×106 and about 1×1014 particles, more generally between about 1×10⁸ and about 1×10¹² particles.

The effectiveness of rAAV delivery can be monitored by several criteria. For example, samples removed by biopsy or surgical excision may be analyzed by in situ hybridization, PCR amplification using vector-specific probes and/or RNAse protection to detect rAAV DNA and/or rAAV mRNA. Also, for example, harvested tissue, joint fluid and/or serum samples can be monitored for the presence of TNF antagonist encoded by the rAAV with immunoassays, including, but not limited to, immunoblotting, immunoprecipitation, immunohistology and/or immunofluorescent cell counting, or with function-based bioassays dependent on TNF antagonist-mediated inhibition of TNF activity. For example, when the rAAV encoded TNF antagonist is a TNFR polypeptide, the presence of the encoded TNFR in harvested samples can be monitored with a TNFR immunoassay or a function-based bioassay dependent on TNFR-mediated inhibition of TNF killing of mouse L929 cells. Examples of such assays are known in the art and described herein.

The invention also provides methods in which administration of rAAV vectors described herein use ex vivo strategies for delivery of polynucleotides to the target joint of the individual. Such methods and techniques are known in the art. See, for example, U.S. Pat. No. 5,399,346. Generally, cells are transduced by the rAAV vectors in vitro and then the transduced cells are introduced into the target joint of the individual. Suitable cells are known to those skilled in the art and include autologous cells, such as stem cells.

The effectiveness of the methods provided herein may, for example, be monitored by assessment of the relative levels of TNF in harvested tissue, joint fluid and/or serum subsequent to delivery of the rAAV vectors described herein. Assays for assessing TNF levels are known in the art and include, but are not limited to, immunoassays for TNF, including, but not limited to, immunoblot and/or immunoprecipitation assays, and cytotoxicity assays with cells sensitive to the cytotoxic activity of TNF. See, for example, Khabar et al., 1995, Immunol. Lett. 46:107-110.

The treated individual may also be monitored for clinical features which accompany the TNF-associated disorder. For example, subjects may be monitored for reduction in signs and symptoms associated with inflammation. For example, after treatment of RA in a subject using methods of the present invention, the subject may be assessed for improvements in a number of clinical parameters including, but not limited to, joint swelling, joint tenderness, morning stiffness, pain, erythrocyte sedimentation rate, and c-reactive protein.

An enhanced treatment effective may also be demonstrated by an extension of the time period between the worsening of the signs or symptoms of the disease, for example a worsening of tenderness or swelling of the target joint, requiring repeat administration of the rAAV vector of the present invention.

The selection of a particular composition, dosage regimen (i.e., dose, timing and repetition) and route of administration depend on a number of different factors, including, but not limited to, the individual's medical history and features of the condition and the individual being treated. The assessment of such features and the design of an appropriate therapeutic regimen is ultimately the responsibility of the prescribing physician. The particular dosage regimen may be determined empirically.

The foregoing description provides, inter alia, compositions and methods for reducing the levels of TNF in an individual or for treating inflammatory arthritis in an individual, comprising administering to the individual an effective amount of an recombinant AAV (rAAV) vector comprising a polynucleotide encoding a TNF antagonist, wherein the individual is being treated systemically with an art recognized effective amount of a polypeptide TNF-α antagonist but still has one or more persistent symptomatic joints despite the systemic polypeptide TNF-α antagonist treatment. It is understood that variations may be applied to these methods by those of skill in this art without departing from the spirit of this invention.

The examples presented below are provided as a further guide to a practitioner of ordinary skill in the art, and are not meant to be limiting in any way.

### EXAMPLES

### Example 1

### Clinical Trial

Study Design The purpose of this study is to evaluate repeat doses of the rAAV vector containing the polynucleotide encoding TNFr:Fc administered to persistently symptomatic joints in individuals with and without concurrent systemic polypeptide TNF-α antagonist therapy. Individuals enrolled in the first cohort receive a dose 1×10¹¹ DRP per mL of joint volume. Individuals are dosed in the second and third cohorts respectively at 1×10¹² and then to 1×10¹³ DRP per mL of joint. If no safety concerns arise after the first three cohorts of 20 individuals each enrolled, 60 additional individuals are randomized into one of three cohorts and receive the rAAV vector containing the polynucleotide encoding TNFr:Fc at one of the three doses' above.

The study design is summarized in Table 1 below. Target joints are assessed for tenderness and swelling every 4-6 weeks.

TABLE 1
Clinical Trial Design

| | Number | Dose | Segment A¹ | Segment B² |
|---|---|---|---|---|
| | of | Concentration | (1^{st} Dose) | (2^{nd} Dose) |
| | Individ- | of | Number of | Number of |
| Cohort | uals | rAAV | Active°:Placebo⁺⁺ | Active° |
| 1* | 20 | 1 × 10¹¹ DRP/mL | 15:5 | 20 |

Pause for DMC review

| | | | | |
|---|---|---|---|---|
| 2* | 20 | 1 × 10¹² DRP/mL | 15:5 | 20 |

Pause for DMC review

| | | | | |
|---|---|---|---|---|
| 3* | 20 | 1 × 10¹³ DRP/mL | 15:5 | 20 |

Pause for DMC review

| | | | | |
|---|---|---|---|---|
| 4⁺ | 20 | 1 × 10¹¹ DRP/mL | 15:5 | 20 |
| 5⁺ | 20 | 1 × 10¹² DRP/mL | 15:5 | 20 |
| 6⁺ | 20 | 1 × 10¹³ DRP/mL | 15:5 | 20 |

¹Segment A is the randomized, double-blind, placebo-controlled portion of the study. Individuals are randomized in a 3:1 ratio to receive an intra-articular injection of tgAAC94 at one of three dose concentrations or placebo.
²Segment B is the open label portion of the study. All individuals enrolled in Segment A are followed until swelling in the target joint reaches predetermined criteria for re-injection (on or after Week A12), or until Week A30, whichever comes first. At that point, each individual is entered in Segment B and receives an intra-articular injection of tgAAC94 at the same dose concentration of their original cohort. Criteria for transition of individuals to Segment B to receive the open-label injection of study drug are based on the degree of swelling of the target joint. If the swelling is at baseline (Day A0) or worse at a study visit on or after Week A12, the subject is eligible to enter Segment B and is scheduled for re-injection within 14 days.
*Cohorts 1-3: The first three individuals dosed in each segment (A and B) are observed for three days each after study administration prior to dosing the next individual in the respective segment of that cohort. The remaining individuals in the respective cohort and segment are dosed without any protocol-specified delays. Enrollment pause after the last subject in each cohort complete the Week A4 visit to allow for a DMC review of cumulative safety data prior to enrollment of individuals in the following cohort(s).
⁺Cohorts 4-6: If no safety concerns arise in Cohorts 1-3, 60 individuals are randomized into Cohorts 4-6 simultaneously. If safety concerns arise in Cohort 3, Cohort 6 may be eliminated.

rAAV Vectors and Placebo Administered to Individuals

For the purposes of this example the rAAV vector is an AAV serotype 2 vector containing the polynucleotide of FIG. 2 encoding the polypeptide TNFr:Fc of FIG. 1. The rAAV is supplied as a frozen sterile formulation in 2 mL vials. The rAAV vector is formulated in a sterile isotonic buffered salt solution containing sodium chloride, glucose, potassium phosphate, calcium chloride, magnesium chloride, and HEPES buffer. Placebo consists of the sterile isotonic buffered salt solution containing sodium chloride, glucose, potassium phosphate, calcium chloride, magnesium chloride, and HEPES buffer that is used to formulate the rAAV vector. Placebo is supplied as a frozen, sterile formulation in 2 mL vials. Each vial contains 1 mL of formulation buffer. The vials are identical in appearance to the vials containing the rAAV vector.

Volume and Dosage of rAAV Administered to Target Joint

The volume of rAAV vector injected in the target joint depends on the joint selected for injection. For the current example the volume is determined based on current clinical practice with intra-articular injections of steroids in patients with inflammatory arthritis. Knees were injected with 5 mL, ankles with 2 mL, elbows with 1.5 mL, wrists with 1 mL, and metacarpophalangeal (MCP) joints with 0.5 mL. Doses administered per cohort are described in Table 2. Dose levels are not blinded for Cohorts 1-3. In contrast, dose levels are blinded for Cohorts 4-6.

TABLE 2
Intra-articular Dosing of rAAV

| | | Cohorts 1 & 4 | Cohorts 2 & 5 | Cohorts 3 & 6 |
|---|---|---|---|---|
| | | (1 × 10¹¹ | (1 × 10¹² | (1 × 10¹³ |
| | Volume of | DRP/mL | DRP/mL | DRP/mL |
| | Injection | joint volume) | joint volume) | joint volume) |
| Joint | (mL) | Dose (DRP) | Dose (DRP) | Dose (DRP) |
| Knee | 5 | 5 × 10¹¹ | 5 × 10¹² | 5 × 10¹³ |
| Ankle | 2 | 2 × 10¹¹ | 2 × 10¹² | 2 × 10¹³ |
| Elbow | 1.5 | 1.5 × 10¹¹ | 1.5 × 10¹² | 1.5 × 10¹³ |
| Wrist | 1 | 1 × 10¹¹ | 1 × 10¹² | 1 × 10¹³ |
| MCP | 0.5 | 0.5 × 10¹¹ | 0.5 × 10¹² | 0.5 × 10¹³ |

### Entrance Criteria of Individuals in the Study

The individuals treated consisted of adults with inflammatory arthritis (RA, PsA or AS as diagnosed according to the published criteria (Arnett et al., 1988; Moll and Wright, 1973; van der Linden et al., 1984)) with persistent moderate (grade 2) or severe (grade 3) swelling in one or more joints eligible for injection, but without disease severe enough to warrant a change in regimen for inflammatory arthritis in next three months. For individuals on disease modifying antirheumatic drugs (DMARDs), individuals must have been on a stable regimen for inflammatory arthritis for the previous three months, with no changes in doses in the four weeks prior to screening. Individuals with RA must have had an adequate trial of at least one DMARD prior to screening. Swelling is graded independently according to a four-point scale, ranging from 0-none, 1-mild, 2-moderate, to 3-severe. The following guidelines from the Dictionary of Rheumatic Diseases should be used to determine the grades of swelling (American Rheumatism Association, 1988):

| Grade | Swelling |
|---|---|
| 0-none | 0 = no swelling |
| 1-mild | 1 = swelling just |
| | appreciable |
| 2-moderate | 2 = swelling but within |
| | normal joint contours |
| 3-severe | 3 = distention by swelling |
| | outside normal joint |
| | contours |

Individuals maintain their usual therapy for inflammatory arthritis and other medical problems. The use of all medications, including over-the-counter medication and treatments is recorded. All changes in concurrent medication during the study period is recorded. If an individual experiences a flare in their inflammatory arthritis that requires a major change in the medical regimen for arthritis, including addition of a DMARD or intra-articular steroid injection in the target joint, the subject is withdrawn from the study.

Administration of rAAV

Joint aspiration, to remove as much synovial fluid as possible, is performed and the rAAV is administered via intra-articular injection at the dose specified using aseptic technique and universal precautions.

### Assessment of Enhanced Therapeutic Effect; Changes in Tenderness and Swelling of Target Joint

Tenderness and swelling of target joints are graded independently every four to six weeks according to a four-point scale, ranging from 0-none, 1-mild, 2-moderate, to 3-severe for both tenderness and swelling according to guidelines from the Dictionary of Rheumatic Diseases (American Rheumatism Association, 1988). Data are presented as a composite score of the tenderness and swelling with the scale of 0-6 representing a maximal number of 3 for severe tenderness and 3 for maximal swelling giving a total potential score of 6 for the most severely affected joints.

Guidelines from the Dictionary of Rheumatic Diseases (American Rheumatism Association, 1988)

| Grade | Tenderness | Swelling |
|---|---|---|
| 0-none | 0 = no tenderness | 0 = no swelling |
| 1-mild | 1 = complaint of tenderness | 1 = swelling just |
| | | appreciable |
| 2-moderate | 2 = complaint of tenderness | 2 = swelling but within |
| | with wincing | normal joint contours |
| 3-severe | 3 = wincing with attempt to | 3 = distention by swelling |
| | withdraw | outside normal joint |
| | | contours |

Improvement is defined as a one or more point decrease in swelling from baseline and is represented by a negative change from baseline in the Mean Tenderness &Swelling (T&S) scores. Worsening is defined as a one or more point increase in the change from baseline of the T&S Score.

### Expanded Assessment of Enhanced Therapeutic Effect of Target Joint

Cohorts 4-6 undergo an expanded panel of target joint assessments. These additional assessments include:
Patient assessment of target joint, consisting of a brief questionnaire addressing overall symptoms, function, and satisfaction with response to study drug injection on appropriate visual-analog scales.
Functional assessment of the target joint, using a modification of the Disabilities of the Arm, Shoulder and Hand (DASH) (Adams et al., 2004; Hudak et al., 1996; Navsarikar et al., 1999) for individuals whose target joint is in the upper extremity, and a modification of the Rheumatoid Arthritis Outcome Score (RAOS) (Bremander et al., 2003) for individuals whose target joint is in the lower extremity.
Repeat assessment of the tenderness and swelling of the target joint, using the four-point scales outlined above, by a second, qualified examiner, to determine the inter-observer variability in assessing the tenderness and swelling of a single joint.

### Joint Inflammation and Damage as Assessed by Magnetic Resonance Imaging (MRI)

MRI scans of the target joint are performed at selected sites, with the goal to perform MRI scans on at least 50% of individuals enrolled in Cohorts 4-6. Joint inflammation and damage are assessed using the Outcome Measures in Rheumatology Clinical Trials (OMERACT) RA MRI scoring system (RAMRIS) (Ostergaard et al., 2005; Ostergaard et al., 2003). The RAMRIS scoring system has been well-validated for use in assessing wrist and MCP joints in RA, and are applied to other joints and other forms of inflammatory arthritis to assess its potential utility as an outcome measure for other joints and inflammatory arthritides.

In accordance with OMERACT RAMRIS guidelines, MRI scans are performed using a core set of basic MRI sequences that includes: (1) imaging in two planes (can be acquired by obtaining a two-dimensional sequence in two planes, or a three dimensional sequence with isometrical voxels in one plane allowing reconstruction in other planes) with T1 weighted images before and after intravenous gadolinium contrast and (2) a T2 weighted fat saturated sequence or, if the latter is not available, a STIR (short tau inversion recovery) sequence. A standardized protocol is developed and used across all sites performing MRI scans.

MRI scans are evaluated in a centralized location by qualified radiologists blinded to treatment assignment. Joint pathology is defined as follows by a modification of the OMERACT 2002 RAMRIS scoring system is used to rate the synovitis, bone erosions, bone edema, joint effusion, and tenosynovitis as described above.
Synovitis: An area in the synovial compartment that shows above normal post-gadolinium enhancement of a thickness greater than the width of the normal synovium (scored on a scale of 0 to 3).
MRI bone erosion: A sharply marginated bone lesion, with correct juxta-articular localization and typical signal characteristics, which if visible in two planes with a cortical break seen in at least one plane (scored on a scale of 0 to 5).
MRI bone edema: A lesion within the trabecular bone, with ill-defined margins and signal characteristics consistent with increased water content (scored on a scale of 0 to 5).

In addition, the following parameters is assessed:
Joint effusion: Characterized as fluid within the joint space(s) of the anatomic region of interest (scored on a scale of 0 to 5).
Tenosynovitis: Defined as fluid surrounding (or within) a tendon adjacent to the anatomic region of interest (scored on a scale of 0 to 4)

### Assessment of Disease Activity

The following assessments are used to assess disease activity in individuals administered the rAAV to the target joint:

Patient's global assessment, on a visual analog scale of 0 (asymptomatic) to 10 (severe symptoms)
Patient's assessment of pain, on a visual analog scale of 0 (no pain) to 10 (severe pain)
Patient's assessment of disability, via a domain of the Health Assessment Questionnaire
Bath Ankylosing Spondylitis Functional Index (BASFI) (AS individuals only)
Bath Ankylosing Spondylitis Disease Activity Index (BASDAI) (AS individuals only)
Physician's global assessment, on a visual analog scale of 0 (asymptomatic) to 10 (severe symptoms)
28-joint count of tender and swollen joints
Erythrocyte sedimentation rate
C-reactive protein level
ACR 20 (RA or PsA Individuals only). If appropriate, the corresponding ACR 50 and ACR 70 are determined in a similar manner.
Modified Disease Activity Score (DAS) (RA individuals only), developed by the European League Against Rheumatism (EULAR) (van Riel and van Gestel, 2000; van Riel et al., 1996).
Assessments in Ankylosing Spondylitis 20 percent response (ASAS 20)(AS Individuals only)
Bath Ankylosing Spondylitis Functional Index (BASFI) (Calin et al., 1994)
Bath Ankylosing Spondylitis Disease Activity Index (BASDAI) (Garrett et al., 1994).

### 7.1.5 Synovial Fluid TNFR:Fc Protein Levels

Synovial fluid is obtained from individuals whose target joints have obvious effusions. Synovial fluid TNFR:Fc protein levels are determined to assess baseline levels in individuals on etanercept and to assess expression of TNFR:Fc in the joint.
Serum TNFR:Fc protein level
Serum anti-AAV2 capsid neutralizing antibodies
Serum Anti-AAV2 Capsid Neutralizing Antibodies
T-cell Responses to AAV2 Capsid

All individuals who receive study agent are included in the analysis.

### Results

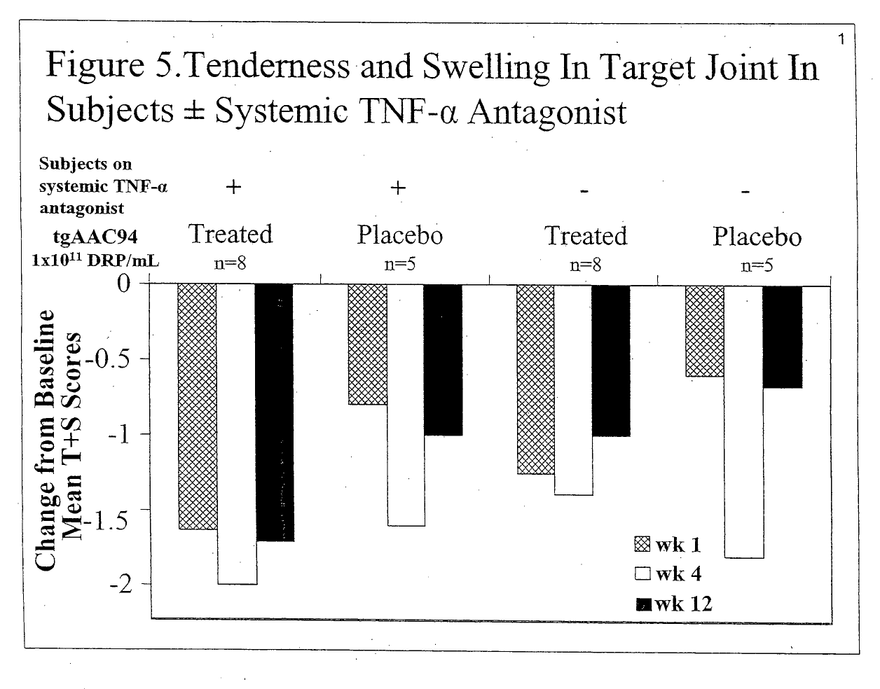

Results from grouped aggregate data in individuals administered 1×10¹¹ DRPs of a rAAV vector containing a polynucleotide encoding TNFr:Fc via intra-articular injection of joints with or without concurrent polypeptide TNF antagonist therapy at weeks 1, 4, and 12 are presented in FIG. 5. Data presented represent a change in baseline tenderness and swelling indices (T&S) measured as described herein. Baseline (T&S) scores ranged from 4.3-4.8 in the example presented. The data demonstrate that there is an enhanced treatment effect of target joints receiving 10¹¹ DRPs of the rAAV vector containing a polynucleotide encoding TNFr:Fc for individuals receiving concurrent polypeptide TNF antagonists at 12 weeks post intra-articular administration compared to joints of individuals receiving the rAAV vector containing the polynucleotide encoding TNFr:Fc not being treated concurrently with polypeptide TNF antagonists. The data also demonstrate that there is an enhanced treatment effect of target joints of individuals receiving 10¹¹ DRPs/ml of the rAAV vector and receiving concurrent polypeptide TNF antagonists at 12 weeks post intra-articular administration of the rAAV vector compared to the joints of individuals receiving only the polypeptide TNF antagonist treatment. Data analysis also demonstrates a prolongation of time to repeat delivery of the rAAV vector.

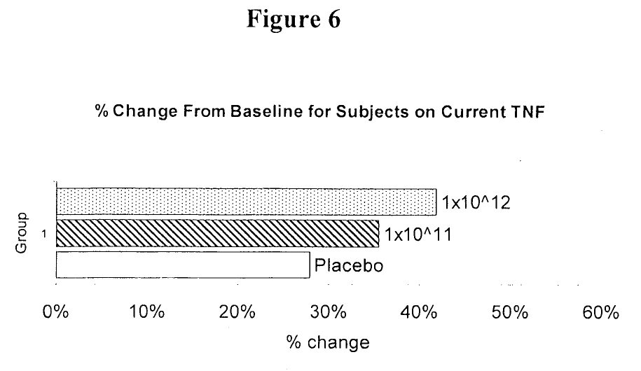

Results from grouped aggregate data in individuals administered 1×10¹¹ or 1×10¹² DRPs of a rAAV vector containing a polynucleotide encoding TNFr:Fc via intra-articular injection of joints with concurrent polypeptide TNF antagonist therapy at week 12 is presented in FIG. 6. Data presented represent the change at week 12 of tenderness and swelling indices (T&S) measured as described herein compared to the baseline tenderness and swelling indices at week 0. Baseline (T&S) scores ranged from 4.3-5.0 in the example presented. The data demonstrate that there is an enhanced treatment effect of target joints receiving either 10¹¹ DRPs or 10¹² DRPs of the rAAV vector containing a polynucleotide encoding TNFr:Fc for individuals receiving concurrent polypeptide TNF antagonists at 12 weeks post intra-articular administration compared to placebo.

### REFERENCES

Arnold E L, Khanna D, Paulus H, et al. Acute injection site reaction to intraarticular etanercept administration. Arthritis Rheum 48:2078-9, 2003.
American Rheumatism Association. Appendix A—Joint Examination. In: Dictionary of the Rheumatic Diseases, Volume 1: Signs and Symptoms. 3rd ed. New York: Contact Associates International, Ltd.; 1988:75-6.
Anderson J J, Baron G, van der Heijde D, et al. Ankylosing spondylitis assessment group preliminary definition of short-term improvement in ankylosing spondylitis. Arthritis Rheum 44:1876-86, 2001.
Arnett F C, Edworthy S M, Bloch D A, et al. The American Rheumatism Association 1987 revised criteria for the classification of rheumatoid arthritis. Arthritis Rheum 31:315-24, 1988.
Bliddal H, Qvistgaard E, Terslev L, et al. Injection of etanercept into arthritis joints: dose-response and efficacy. Arthritis Rheum 46 Suppl 9:S518-9, 2002.
Bliddal H. Intraarticular injection of anti-tumor necrosis factor: Comment on the letter by Arnold et al. Arthritis Rheum 50:2037-8, 2004.
Braun J and Sieper J. Biological therapies in the spondyloarthritides—the current state. Rheumatology 43:1072-84, 2004.
Bremander A B, Petersson I F and Roos E M. Validation of the Rheumatoid and Arthritis Outcome Score (RAOS) for the lower extremity. 2003 Oct. 17; 1(1):55. Health Qual Life Outcomes: 55, 2003.
Calin A, Garrett S, Whitelock H, et al. A new approach to defining functional ability in ankylosing spondylitis: the development of the Bath Ankylosing Spondylitis Functional Index. J Rheumatol 21:2281-5, 1994.
Chan J M, Villarreal G, Jin W W, et al. Intraarticular gene transfer of TNFR:Fc suppresses experimental arthritis with reduced systemic distribution of the gene product. Mol Ther 6:727-36, 2002.
Criscione L G and St Clair E W. Tumor necrosis factor-alpha antagonists for the treatment of rheumatic diseases. Curr Opin Rheumatol 14:204-11, 2002.
FDA Briefing Document. Update on the TNF-a blocking agents. Mar. 4, 2003 Meeting of the Arthritis Advisory Committee, 2003.
Felson D T, Anderson J J, Boers M, et al. American College of Rheumatology. Preliminary definition of improvement in rheumatoid arthritis. Arthritis Rheum 38:727-35, 1995.
Gardner G C. Inflammatory arthritis in the era of the biologics. Clin Appl Immunol Rev 5:19-44, 2005.
Garrett S, Jenkinson T, Kennedy L G, et al. A new approach to defining disease status in ankylosing spondylitis: the Bath Ankylosing Spondylitis Disease Activity Index. J Rheumatol 21:2286-91, 1994.
Hochberg M C, Chang R W, Dwosh I, et al. The American College of Rheumatology 1991 revised criteria for the classification of global functional status in rheumatoid arthritis. Arthritis Rheum 35:498-502, 1992.
Hudak P L, Amadio P C and Bombardier C. Development of an upper extremity outcome measure: the DASH (disabilities of the arm, shoulder and hand) [corrected]. The Upper Extremity Collaborative Group (UECG). Am J Ind Med 29:602-8, 1996.
Moll J M and Wright V. The pattern of chest and spinal mobility in ankylosing spondylitis. An objective clinical study of 106 patients. Rheumatol Rehabil 12:115-34, 1973.

Moll J M and Wright V. Psoriatic arthritis. Semin Arthritis Rheum 3:55-78, 1973. Navsarikar A, Gladman D D, Husted J A, et al. Validity assessment of the disabilities of arm, shoulder, and hand questionnaire (DASH) for patients with psoriatic arthritis. J Rheumatol 26:2191-4, 1999.
Osborn T G. Intraarticular etanercept versus saline in rheumatoid arthritis: a single injection double blind placebo controlled study. Arthritis Rheum 46 Suppl 9:S518, 2002.
Ostergaard M, Peterfy C, Conaghan P, et al. OMERACT Rheumatoid Arthritis Magnetic Resonance Imaging Studies. Core set of MRI acquisitions, joint pathology definitions, and the OMERACT RA-MRI scoring system. J Rheumatol 30:1385-6, 2003.
Ostergaard M, Edmonds J, McQueen F, et al. An introduction to the EULAR-OMERACT rheumatoid arthritis MRI reference image atlas. Ann Rheum Dis 64 Suppl 1:13-7, 2005.
van der Linden S, Valkenburg H A and Cats A. Evaluation of diagnostic criteria for ankylosing spondylitis. A proposal for modification of the New York criteria. Arthritis Rheum 27:361-8, 1984.
van Riel P L, van Gestel A M and van de Putte L B. Development and validation of response criteria in rheumatoid arthritis: steps towards an international consensus on prognostic markers. Br J Rheumatol 35 Suppl 2:4-7, 1996.
van Riel P L and van Gestel A M. Clinical outcome measures in rheumatoid arthritis. Ann Rheum Dis 59 Suppl 1:128-31, 2000.
Enbrel (etanercept). Physicians' Desk Reference, Thomson Healthcare, 2005. (Accessed Mar. 18, 2005, at http://www.pdr.net/pdmet/librarian.)

1. A method for treating inflammatory arthritis in an individual, comprising administering to a persistently symptomatic joint of the individual an effective amount of an recombinant AAV (rAAV) vector comprising a polynucleotide encoding a fusion polypeptide comprising an extracellular domain of tumor necrosis factor receptor (TNFR) and a constant domain of an immunoglobulin molecule, wherein the individual has been treated systemically with an art recognized effective amount of a polypeptide TNF-α antagonist but still has one or more persistently symptomatic joints despite the systemic polypeptide TNF-α antagonist treatment.

2. The method of claim 1, wherein the rAAV vector is administered locally or regionally to the joint.

3. The method of claim 1, wherein the rAAV vector is administered by intra-articular injection.

4. The method of claim 1, wherein the rAAV vector is administered in conjunction with the polypeptide TNF-α antagonist.

5. The method of claim 1, wherein the polypeptide TNF-α antagonist is selected from the group consisting of a soluble TNF receptor, an anti-TNF-α monoclonal antibody, and a soluble IL-1 receptor.

6. The method of claim 1, wherein the polypeptide TNF-α antagonist is selected from the group consisting of etanercept, infliximab, adalimumab, and Anakinra.

7. The method of claim 1, wherein the TNFR extracellular domain is from p75 TNFR.

8. The method of claim 1, wherein the polynucleotide encoding the TNFR polypeptide is operably linked to a heterologous promoter.

9. The method of claim 1, wherein the polynucleotide encoding the TNFR polypeptide is operably linked to a constitutive promoter.

10. The method of claim 1, wherein the polynucleotide encoding the TNFR polypeptide is operably linked to an inducible promoter.

11. The method of claim 9, wherein the inducible promoter is from the TNFα gene.

12. A method for enhancing the treatment effect of a polypeptide TNF-α antagonist in an individual, comprising administering to a persistently symptomatic joint of the individual an effective amount of a recombinant AAV (rAAV) vector comprising a polynucleotide encoding a fusion polypeptide comprising an extracellular domain of tumor necrosis factor receptor (TNFR) and a constant domain of an immunoglobulin molecule, in conjunction with the polypeptide TNF-α antagonist, wherein the individual has been treated systemically with the polypeptide TNF-α antagonist but still has one or more persistently symptomatic joints despite the systematic polypeptide TNF-α antagonist treatment.

13. The method of claim 12, wherein the rAAV vector is administered locally or regionally to the joint.

14. The method of claim 12, wherein the rAAV vector is administered by intra-articular injection.

15. The method of claim 12, wherein the polypeptide TNF-α antagonist is selected from the group consisting of a soluble TNF receptor, an anti-TNF-α monoclonal antibody, and a soluble IL-1 receptor.

16. The method of claim 12, wherein the polypeptide TNF-α antagonist is selected from the group consisting of etanercept, infliximab, adalimumab, and Anakinra.

17. The method of claim 12, wherein the TNFR extracellular domain is from p75 TNFR.

18. The method of claim 12, wherein the polynucleotide encoding the TNFR polypeptide is operably linked to a heterologous promoter.

19. The method of claim 12, wherein the polynucleotide encoding the TNFR polypeptide is operably linked to a constitutive promoter.

20. The method of claim 12, wherein the polynucleotide encoding the TNFR polypeptide is operably linked to an inducible promoter.

21. The method of claim 20, wherein the inducible promoter is from the TNFα gene.
